(19) United States
(12) Patent Application Publication
(10) Pub. No.: US 2012/0165230 A1
(43) Pub. Date: Jun. 28, 2012

(54) BIOLOGICALLY PRODUCED CYCLIC AFFINITY TAGS

(75) Inventors: Tjibbe Bosma, Lippenhuizen (NL); Anneke Kuipers, Haule (NL); Rick Rink, Groningen (NL); Gert N. Moll, Groningen (NL)

(73) Assignee: LanthioPep B.V., Groningen (NL)

(21) Appl. No.: 13/376,577

(22) PCT Filed: Jun. 23, 2010
(86) PCT No.: PCT/NL2010/050389

§ 371 Date: Feb. 21, 2012

(30) Foreign Application Priority Data
Jun. 24, 2009 (EP) 09163581.3

(51) Int. Cl. C40B 40/10; C07K 14/00; C12P 21/00

(52) U.S. Cl. 506/18; 435/68.1; 530/350

(57) ABSTRACT

Described are novel ways of introducing an affinity tag into a protein of interest. Provided is an enzymatic method for providing a proteinaceous substance comprising a polypeptide of interest and a cyclic affinity tag, comprising: (a) providing at least one precursor proteinaceous substance, the precursor comprising the protein of interest and at least one motif of the general formula X1-Tag-X2, wherein X1 and X2 represent amino acids whose side chains can be linked enzymatically by a covalent bond; Tag is a short amino acid sequence capable of binding to a binding partner of the tag when cyclized; (b) contacting the precursor with at least one enzyme capable of forming a covalent bond between X1 and X2, thereby introducing an intramolecular ring structure comprising the Tag sequence; and (c) isolating the resulting cyclized proteinaceous substance. Also provided are proteinaceous substances obtainable thereby and the use thereof, for instance, for preparing a peptide library.

### CROSS-REFERENCE TO RELATED APPLICATIONS

This is a national phase entry under 35 U.S.C. §371 of International Patent Application PCT/NL2010/050389, filed Jun. 23, 2010, published in English as International Patent Publication WO 2010/151126 A1 on Dec. 29, 2010, which claims the benefit under Article 8 of the Patent Cooperation Treaty to European Patent Application Serial No. 09163581.3, filed Jun. 24, 2009.

### TECHNICAL FIELD

The invention relates to the field of biotechnology and recombinant protein expression, purification and immobilization. In particular, it relates to novel ways of introducing an affinity tag into a protein of interest.

### BACKGROUND

A wide variety of affinity tags have been developed and are used throughout biotechnology. The widespread success of affinity tags throughout the biological sciences has prompted interest in developing new and convenient labeling strategies. Affinity tags are well-established tools for capturing a recombinant protein, e.g., in immobilization and/or purification procedures. More recently, these tags have been utilized for selective biological targeting towards multiplexed protein detection in numerous imaging applications as well as for drug delivery. The most commonly employed affinity tags range from short polypeptide sequences to whole proteins, which can confer advantageous solubility effects. For example, small peptide epitopes such as polyhistidine tags, which can bind to immobilized metal chelates, as well as the myc-tag and FLAG-tag, which can bind to immobilized antibodies, are commonly used for the isolation and immobilization of recombinant proteins.

Another small peptide epitope that has gained wide use is the streptavidin-specific Strep-tag (Schmidt et al., Protein Eng. 6 (1993) 109-122; US 2006/0106199 and U.S. Pat. No. 6,841,359). Streptavidin-binding peptide sequences have been discovered by screening peptide libraries, most, but not all, of which contain the His-Pro-Gln (HPQ) motif. The development of streptavidin-fusion peptides has aided in a variety of unique biochemical applications and has made streptavidin, the non-glycosylated bacterial relative of avidin, the preferred protein in many applications of the (strept)avidin-biotin technologies (Keefe et al., Protein Expr. Purif. 23 (2001) 440-446; Lamla et al., Protein Expr. Purif. 33 (2003) 39-47). Small peptides such as the Strep-tag can easily be expressed as fusions with larger proteins for use in purification or other conjugation applications. The availability of labeled streptavidin, as well as streptavidin immobilized on solid supports, has made these peptides extremely useful. Using streptavidin as a model receptor system, it was found that disulfide-constrained cyclic peptide strep motifs bind streptavidin with affinities up to three orders of magnitude higher than the corresponding linear sequences (Giebel et al., Biochemistry 1995, 34, 15430-15435). Katz et al. (J. Am. Chem. Soc. 1995, 117, 8541-8547) designed and chemically synthesized cyclic streptavidin-binding peptides comprising the motif CHPQGPPC, wherein the disulfide is replaced by a thioether cross-link. The thioether-cross-linked peptides were reported to display enhanced stability as compared to their disulfide counterpart.

### SUMMARY

Thus, cyclic affinity tags like cyclized strep motifs have advantageous properties for use as a protein tag over their linear counterparts. However, in contrast to fusion proteins comprising linear proteins that can simply be obtained by recombinant protein expression, the provision of a protein comprising a cyclic affinity tag typically requires chemical modification, which is highly undesirable in terms of time, efforts and costs involved. The spontaneous formation of disulfide bridges usually lacks specificity, while the bridges themselves lack stability.

The inventors, therefore, set out to provide alternative methods for providing a protein of interest with a cyclized affinity tag. It was found that a linear tag sequence can be cyclized biologically, i.e., non-chemically, if the tag sequence is flanked on each side by amino acids capable of forming together a covalent bond upon enzyme action, e.g., by an enzyme present in a host cell expressing a construct encoding the protein of interest comprising the tag sequence.

Therefore, disclosed is a method for providing a proteinaceous substance comprising a protein of interest and a cyclic affinity tag, comprising the steps of:
a) providing at least one precursor proteinaceous substance, the precursor comprising the protein of interest and at least one motif of the general formula X1-Tag-X2 wherein X1 and X2 represent amino acids whose side chains can be linked enzymatically by a covalent bond; Tag is an amino acid sequence capable of binding to a (proteinaceous) binding partner when cyclized;
b) contacting the precursor with at least one enzyme capable of forming a covalent bond between X1 and X2, thereby introducing an intramolecular ring structure comprising the Tag sequence; and
c) isolating the resulting cyclized proteinaceous substance.

Thus, disclosed is a method involving enzyme-mediated cyclization. As used herein, the expression “enzyme-mediated cyclization” is meant to indicate that at least one step required for ring formation is performed enzymatically, i.e., non-chemically. The enzymatic step may be performed in vivo (by a non-human or non-animal host cell) or in vitro. The enzyme-mediated step(s) may include enzymatic modification of amino acid residues (e.g., dehydration), and/or enzymatic closure of the ring. Enzymatic cyclization may consist of just the enzymatic formation of highly reactive dehydroalanine, but can also consist of cyclase-action (R. Rink, 2007, Biochemistry 46:13179-13189).

In particular, provided is an enzymatic method for providing a proteinaceous substance comprising a polypeptide of interest and a cyclic affinity tag, comprising the steps of:
a) providing at least one precursor proteinaceous substance, the precursor comprising the protein of interest and at least one motif of the general formula X1-Tag-X2 wherein
   X1 and X2 represent amino acids whose side chains can be linked by a lantibiotic enzyme capable of forming a thioether bridge between residues X1 and X2;
   Tag is an amino acid sequence serving as affinity tag when cyclized, the affinity tag allowing for capture of the proteinaceous substance to a specific binding partner of the tag,
   and wherein the motif is preceded N-terminally by a lantibiotic leader sequence;
b) contacting the precursor with at least one lantibiotic enzyme, allowing for the formation of a thioether bridge between X1 and X2, thereby introducing an intramolecular ring structure comprising the Tag sequence; and
c) isolating the resulting cyclized proteinaceous substance.

In one embodiment, steps a) and b) are performed in a host cell comprising at least one enzyme capable of forming a covalent bond between X1 and X2, the host cell being provided with a nucleic acid molecule encoding the precursor proteinaceous substance. It is also possible to provide the precursor proteinaceous substance by recombinant expression and perform the ring closure by contacting the substance with the appropriate enzyme(s) in vitro. Step c) advantageously comprises using an immobilized binding partner, like an antibody or other proteinaceous substance, of the cyclized Tag sequence. For example, affinity chromatography is suitably used.

The polypeptide of interest can be any proteinaceous molecule, including a biologically active polypeptide, such as a hormone, an antimicrobial peptide, receptor agonist, receptor antagonist, or a receptor-binding peptide without biological effect. The introduction of lantibiotic enzyme-mediated thioether bridge in a (biologically) active non-lantibiotic polypeptide of interest is known in the art. See, e.g., Kluskens et al., J. Pharm. and Exp. Therapeutics 2009, Vol. 328, No. 3; Rink et al., 2007, Biochemistry Vol 46, No. 45, 13179-13189; US 2005/164339. However, a polypeptide comprising a thioether-cyclized affinity tag allowing for specific capture of the polypeptide has heretofore never been disclosed or suggested.

The positioning of the at least one tag sequence (motif) of the general formula X1-Tag-X2 within the proteinaceous substance relative to the polypeptide of interest can vary. In one embodiment, the motif is inserted into the amino acid sequence of the polypeptide of interest (“internal” tag). However, insertion of a foreign sequence can be detrimental for protein function. It may thus be preferred that the motif is added to the polypeptide of interest, e.g., by N- or C-terminal fusion (“external” or “exogenous” tag). In that case, the proteinaceous substance may comprise a cleavage site between the polypeptide of interest and the at least one motif, such that the motif can be removed following step c) by action of the appropriate cleaving enzyme to release the polypeptide of interest. Exemplary cleavage sites included a Factor X or a Glu-C-cleavage site.

In certain embodiments, the proteinaceous substance is a polypeptide of interest wherein a portion is replaced by at least one motif, such that the motif is an integral part of the polypeptide of interest (“intrinsic” tag). As will be described herein, an intrinsic tag can be realized by replacing a stretch of amino acids being part of a naturally occurring intramolecular ring structure with an amino acid sequence that encodes (at least when cyclized) an affinity tag while the replacement leaves the desired propertie(s) of the polypeptide intact. This approach is especially suitable for introducing an affinity tag into a biologically produced thioether-bridged peptide, such as nisin or any other type of lantibiotic. An advantage of an intrinsic affinity tag is that it eliminates the need of costly removal of the tag.

Tag is an amino acid sequence serving as an affinity tag when cyclized. The term “affinity tag” is well known in the art and the skilled person will understand that it refers to an amino acid sequence capable of binding to a (proteinaceous) non-natural binding partner, typically with a dissociation constant in the micromolar range, e.g., less than 10 μM. As used herein, the term “affinity tag” refers to a polypeptide sequence that has affinity for a specific capture reagent and that can be separated from a pool of proteins and thus purified on the basis of its affinity for the binding partner. Thus, the affinity tag allows for capture of the proteinaceous substance of the invention to a specific binding partner. Preferably, the Tag sequence consists of two to twenty amino acid residues, more preferably two to fifteen. Thus, X1 and X2 may be separated by at most twenty, preferably at most fifteen, more preferably two to eight amino acid residues, e.g., 4, 5, 6 or 7 amino acids. As used herein, the term “affinity tag” refers to any sequence capable of binding specifically to a tag-specific binding partner. The binding is typically characterized by a high Kₒₙ and a low K_{off}. The proteinaceous substance that comprises the tag usually is at least 1.5-fold larger, usually two- to eight-fold larger than the tag itself. Although the invention is exemplified with a cyclized Strep-tag, a method of the invention is not restricted to any type of affinity tag allowing for capture of the proteinaceous substance, such that the substance can be purified, isolated and/or immobilized. The other part of the substance, i.e., the “non-tag” part comprising the peptide to be modified, will not bind to the Tag-binding partner but can, of course, have a distinct binding partner, like a receptor or enzyme.

In one embodiment, the Tag sequence comprises or consists of the sequence Arg-Gly-Asp (RGD) (SEQ ID NO:1) and is capable of binding to the glycoprotein IIb/IIIa adhesion molecule. In another embodiment, the Tag sequence is a streptavidin-binding sequence (Strep tag) having a binding affinity for streptavidin of at least submicromolar Kd. For instance, the streptavidin-binding sequence is selected from the group consisting of His-Pro-Gly (HPG) (SEQ ID NO:2), His-Pro-Lys (HPK) (SEQ ID NO:3), His-Pro-Met (HPM) (SEQ ID NO:4), His-Pro-Gln (HPQ) (SEQ ID NO:5), His-Pro-Asn (HPQ) (SEQ ID NO:6) and His-Pro-Gln-Phe (HPQF) (SEQ ID NO:7). In a specific aspect, the streptavidin-binding sequence is His-Pro-Gln (HPQ) (SEQ ID NO:5) or His-Pro-Gln-Phe (HPQF) (SEQ ID NO:7). Other useful Tag sequences include DVEAW (SEQ ID NO:8), DVEAWL/I (SEQ ID NO:9), DVEA (SEQ ID NO:10), VEAW (SEQ ID NO:11), DVE (SEQ ID NO:12), VEA (SEQ ID NO:13), EAW (SEQ ID NO:14), VPLVET (SEQ ID NO:15), DVXAW (SEQ ID NO:16), EPDWF/Y (SEQ ID NO:17), GDF/WXF (SEQ ID NO:18), PWXWL (SEQ ID NO:19), VPEY (SEQ ID NO:20), wherein X is an arbitrary amino acid (see U.S. Pat. No. 6,841,359 and US2008/0032340). Either one of these Tags are advantageously used in a method wherein step c) comprises streptavidin-based affinity chromatography.

The choice of amino acid residues X1 and X2, the residues to form the intracellular ring structure comprising the Tag sequence, will, of course, depend on the enzyme activity/activities to be used. For example, if a lantibiotic-enzyme activity is used for enzymatic ring closure, then X1 and X2 can represent residues whose side chains can be converted to a (methyl)lanthionine bridge. In addition, to allow for recognition of the motif by a lantibiotic enzyme, the motif is preceded by a lantibiotic leader sequence. Accordingly, provided is an enzymatic method for providing a proteinaceous substance comprising a protein of interest and a cyclic affinity tag, comprising the steps of:
a) providing at least one precursor proteinaceous substance, the precursor comprising the protein of interest and at least one motif of the general formula X1-Tag-X2 wherein X1 is selected from Dhb (dehydrobutyrine), Dha (dehydroalanine), Thr, and Ser and wherein X2 is Cys or Lys; or wherein X1 is Cys or Lys and X2 is selected from Dhb, Dha, Thr and Ser; and wherein the motif is preceded N-terminally by a lantibiotic leader sequence. The distance between the leader sequence and the Tag sequence can vary but is typically between 0 and 100 amino acids, preferably between 0 and 50 amino acids, more preferentially between 0 and 20 amino acids;
b) contacting the precursor with at least one lantibiotic enzyme capable of forming a covalent bond between X1 and X2, thereby introducing an intramolecular ring structure comprising the Tag sequence; and
c) isolating the resulting proteinaceous substance comprising a lantibiotic-enzyme-cyclized thioether-bridged affinity tag; the thioether bond being between a D- and an L-amino acid or between an L- and a D-amino acid.

In certain embodiments, X1 is Dha or Dhb and X2 is Cys. Suitable lantibiotic leader sequences are well known in the art and include naturally occurring or genetically engineered lantibiotic leader sequences, for instance, nisin or lacticin3147 leader sequences. Preferred leader sequences for use herein include leader sequences with a conserved FNLD (SEQ ID NO:21) box at positions −18 to −15 counted from the cleavage site and ending with a factor X or GluC cleavage site inside the leader. Alternatively, the leader sequence can have conserved boxes ELD (SEQ ID NO:22) at positions −8 to −6 and EEV (SEQ ID NO:23) at positions −14 to −12 also having a factor X or GluC cleavage site at its C-terminus. Some aligned leader sequences with conserved boxes for use herein are described in US2009042246.

Various possible lantibiotic enzymes may be used to catalyze ring closure. Partially depending on the nature of X1 and X2, the precursor proteinaceous substance may be contacted with lantibiotic enzyme LanM, cyclase LanC (in the case of a combination of a dehydroresidue and a cysteine) or (e.g., in the case wherein X1 or X2 is Ser or Thr) a combination of a lantibiotic dehydratase LanB and cyclase LanC. A method of the invention is efficiently carried out in a host cell comprising one or more lanthionine-generating enzymes. For example, the host cell comprises the proteins LanB; LanC and LanT; LanM and LanT; LanB and LanC; or only LanM. Suitable host cells are Gram-positive bacteria, e.g., Lactococcus lactis, Bacillus cereus, Streptococcus epidermis, Streptomyces lividans or actinomycetes, e.g., Actinoplanes garbadinensis. Preferably, the lantibiotic-producing host transformed with the polynucleic acid encoding the precursor proteinaceous substance is a lacticin3147-producing host or a nisin-producing host. More preferably, the lacticin3147-producing host and the nisin-producing host is a strain of Lactobacillus lactis, NZ9000. When using a host cell with a lantibiotic enzyme system in which the transporter also has the leaderpeptidase activity, it will be relevant not to comprise the recognition of the novel lantibiotic by the self-protection system. In other systems, the leaderpeptidase may be left out of the host cell, thus producing the novel lantibiotic with the leaderpeptide still attached to it, ensuring inactivity of the prelantibiotic and its harmlessness to the producer cell until removal of the leaderpeptide from the harvested prelantibiotic. In vitro synthesis of thioether rings comprising a streptavidin-binding motif by LanM is also possible.

(Methyl)lanthionine-cyclized tag-motif containing polypeptides can be conveniently produced by cells containing a two-plasmid expression system. lanBTC or lanMT could be encoded by one plasmid, for instance, a bidirectionally replicating pIL plasmid. The precursor proteinaceous substance can be encoded by a second plasmid, for instance, a pNZ4048 rolling circle replicating plasmid. Having the modification genes and the polynucleotide encoding the protein to be cyclized on the same plasmid is also possible but less practical. Accordingly, in a specific aspect, the host cell comprises a first vector encoding the precursor proteinaceous substance and a second vector encoding one or more lantibiotic enzyme(s), for example, LanBTC or LanMT. Thus, the invention also relates to a nucleic acid sequence encoding the precursor proteinaceous substance, a vector comprising the nucleic acid sequence, and a host cell provided with the nucleic acid sequence, preferably being part of a suitable expression vector.

A method for providing a protein of interest with a thioether-cyclized affinity tag is especially advantageous if the protein of interest itself also (i.e., in addition to the cyclized affinity tag) comprises at least one thioether-containing intramolecular ring structure. For example, it provides a specific efficient one-step procedure for the purification of a biologically produced thioether-bridged peptide. Another attractive application resides in the purification of lantibiotics, wherein the cyclized tag is present as an intrinsic tag of the lantibiotic. For instance, it provides nisin with a thioether-bridged Strep-tag replacing rings DE. The resulting nisin mutant is readily purified with streptag column. Since nisin A is world-wide applied, a drastically improved production method is of great commercial importance. Advantages over existing (i.e., external) tags used for lantibiotic purification are the high affinity allowing high yield, high purity and making the need for costly removal of external tags obsolete.

Yet another application relates to the generation of thioether-peptide libraries immobilized on chips. Thioether-bridged peptides may have strongly enhanced therapeutic potential (Kluskens et al. (2009) J. of Pharm. and Exp. Therapeutics Vol. 328, 849-854). To obtain stable thioether-bridged peptides, a screening process is generally performed to select for functional peptides. Specific thioether-bridged peptides can be produced in a host cell, like Lactococcus lactis, comprising lantibiotic enzymes. However, in the latter case, the peptides are not physically linked to their DNA, thus posing a hurdle to the identification of the sequence of the selected peptides. By virtue of a method disclosed herein, thioether-bridged peptides comprising a lantibiotic enzyme-introduced thioether affinity tag can be biologically produced. Hence, peptides can be produced, identified and immobilized, e.g., on a defined spot on multiple identical chips provided with a suitable binding partner of the cyclized tag. This allows for the generation of a library with lantibiotic enzyme-cyclized thioether peptides, which can be screened for specific properties, such as binding or kinase-mediated phosphorylation. In a specific aspect, the polypeptide of interest is a member of a library of thioether-bridged peptides, for example a member of a hexa-, hepta- or octapeptide library. The peptides within the library may be screened for a wide variety of properties, including being a kinase substrate or a receptor ligand, such as a Her2-receptor-binding peptide or a G3P-receptor-binding peptide.

In certain embodiments, the motif X1-Tag-X2 is designed to allow for the biological production of a thioether-cyclized streptavidin-binding sequence. To that end, the motif may contain a sequence selected from His-Pro-Gly (HPG) (SEQ ID NO:2), His-Pro-Lys (HPK) (SEQ ID NO:3), His-Pro-Met (HPM) (SEQ ID NO:4), His-Pro-Gln (HPQ) (SEQ ID NO:5) and His-Pro-Gln-Phe (HPQF) (SEQ ID NO:7), which sequence is flanked by X1 and X2 wherein X1 is selected from Dhb, Dha, Thr, and Ser and wherein X2 is Cys or Lys; or wherein X1 is Cys or Lys and X2 is selected from Dhb, Dha, Thr and Ser. Preferably, the motif comprises the streptavidin-binding sequence His-Pro-Gln (HPQ) (SEQ ID NO:5) or His-Pro-Gln-Phe (HPQF) (SEQ ID NO:7). The inventors found that lantibiotic cyclases can catalyze ring closure of Strep-containing sequences (e.g., DhbHPQFC (Dhb-SEQ ID NO:52) and DhbHPQFGC (Dhb-SEQ ID NO:55)). This was unexpected for at least the following reasons: (i) these sequences do not at all occur in natural lantibiotics; (ii) hardly any mutations in nisin's ring C have been published and simultaneous replacement of four residues in one lantibiotic thioether ring has not been reported, (iii) the presence of the helix-breaking residue Pro was expected to reduce the likelihood of ring formation.

The X1-Tag-X2 motif consists, for example, of an amino acid sequence selected from the group consisting of Dha-His-Pro-Gln-Phe-Cys (Dha-SEQ ID NO:48); Dhb-His-Pro-Gln-Phe-Cys (Dhb-SEQ ID NO:48); Ser-His-Pro-Gln-Phe-Cys (SEQ ID NO:24); Thr-His-Pro-Gln-Phe-Cys (SEQ ID NO:25); Cys-His-Pro-Gln-Phe-Dha (SEQ ID NO:49-Dha); Cys-His-Pro-Gln-Phe-Dhb (SEQ ID NO:49-Dhb); Cys-His-Pro-Gln-Phe-Ser (SEQ ID NO:26); Cys-His-Pro-Gln-Phe-Thr (SEQ ID NO:27); Dha-His-Pro-Gln-Cys (Dha-SEQ ID NO:50); Dhb-His-Pro-Gln-Cys (Dhb-SEQ ID NO:50); Ser-His-Pro-Gln-Cys (SEQ ID NO:28); Thr-His-Pro-Gln-Cys (SEQ ID NO:29); Cys-His-Pro-Gln-Dha (SEQ ID NO:51-Dha); Cys-His-Pro-Gln-Dhb (SEQ ID NO:51-Dhb); Cys-His-Pro-Gln-Ser (SEQ ID NO:30); Cys-His-Pro-Gln-Thr (SEQ ID NO:31); Ser-His-Pro-Gln-Phe-Lys (SEQ ID NO:32); Thr-His-Pro-Gln-Phe-Lys (SEQ ID NO:33); Lys-His-Pro-Gln-Phe-Ser (SEQ ID NO:34); Lys-His-Pro-Gln-Phe-Thr (SEQ ID NO:35); Dha-His-Pro-Gln-Phe-Lys (Dha-SEQ ID NO:52); Dhb-His-Pro-Gln-Phe-Lys (Dhb-SEQ ID NO:52); Lys-His-Pro-Gln-Dha (SEQ ID NO:53-Dha); Lys-His-Pro-Gln-Dhb (SEQ ID NO:53-Dhb); Ser-His-Pro-Gln-Lys (SEQ ID NO:36); Thr-His-Pro-Gln-Lys (SEQ ID NO:37); Lys-His-Pro-Gln-Ser (SEQ ID NO:38); Lys-His-Pro-Gln-Thr (SEQ ID NO:39); Dha-His-Pro-Gln-Lys (Dha-SEQ ID NO:54); Dhb-His-Pro-Gln-Lys (Dhb-SEQ ID NO:54); Lys-His-Pro-Gln-Dha (SEQ ID NO:53-Dha); and Lys-His-Pro-Gln-Dhb (SEQ ID NO:53-Dhb). In a preferred embodiment, the sequence is selected from the group consisting of Dha-His-Pro-Gln-Phe-Cys (Dha-SEQ ID NO:48); Dhb-His-Pro-Gln-Phe-Cys (Dhb-SEQ ID NO:48); Ser-His-Pro-Gln-Phe-Cys (SEQ ID NO:24); Thr-His-Pro-Gln-Phe-Cys (SEQ ID NO:25); Cys-His-Pro-Gln-Phe-Dha (SEQ ID NO:49-Dha); Cys-His-Pro-Gln-Phe-Dhb (SEQ ID NO:49-Dhb); Cys-His-Pro-Gln-Phe-Ser (SEQ ID NO:26); Cys-His-Pro-Gln-Phe-Thr (SEQ ID NO:27); Dha-His-Pro-Gln-Cys (Dha-SEQ ID NO:50); Dhb-His-Pro-Gln-Cys (Dhb-SEQ ID NO:50); Ser-His-Pro-Gln-Cys (SEQ ID NO:28); Thr-His-Pro-Gln-Cys (SEQ ID NO:29); Cys-His-Pro-Gln-Dha (SEQ ID NO:51-Dha); Cys-His-Pro-Gln-Dhb (SEQ ID NO:51-Dhb); Cys-His-Pro-Gln-Ser (SEQ ID NO:30) and Cys-His-Pro-Gln-Thr (SEQ ID NO:31).

A further aspect relates to a proteinaceous substance comprising a cyclic affinity tag obtainable hereby. More specifically, the proteinaceous substance comprises a lantibiotic enzyme-mediated cyclized affinity tag. For instance, it comprises a cyclic tag sequence flanked by a dAla-S-Ala, or an Ala-S-dAla or a dAbu-S-Ala or an Ala-S-dAbu or an Ala-N-Lys or a Lys-N-Ala. In contrast to chemically synthesized thioether cross-linked tags wherein the “bridging” amino acids both have the L stereochemistry, the biologically produced thioether-cyclized tags contain either a D,L- or an LD-thioether-linked ring structure bridging. Therefore, in one embodiment, there is provided a proteinaceous substance comprising at least one cyclic tag sequence, the tag sequence being part of a motif containing a D,L- or an LD-thioether-linked ring structure that bridges (in the N- to C-terminal direction) a D-amino acid and an L-amino acid, or an L-amino acid to a D-amino acid. Preferably, the thioether-linked ring structure bridges (in the N- to C-terminal direction) a D-amino acid to an L-amino acid. Also provided is a polynucleotide encoding a proteinaceous substance according to the invention, as well as vectors comprising the polynucleotide. Host cells comprising a polynucleotide or vector according to the invention are also encompassed. These are of particular use for practicing a method of the invention.

As indicated hereinabove, the cyclic tag may be an external (either N- or C-terminal), internal or intrinsic tag. In the case of an external tag, the proteinaceous substance preferably comprises a cleavage site between the polypeptide of interest and the at least one cyclic binding motif. Intrinsic tags are, however, preferred.

In one embodiment, a proteinaceous substance comprising a cyclic affinity tag obtainable by a method of the invention comprises at least one cyclized streptavidin-binding sequence, for instance, selected from the group consisting of His-Pro-Gly (HPG) (SEQ ID NO:2), His-Pro-Lys (HPK) (SEQ ID NO:3), His-Pro-Met (HPM) (SEQ ID NO:4), His-Pro-Gln (HPQ) (SEQ ID NO:5), His-Pro-Gln-Phe (HPQF) (SEQ ID NO:7). Lanthionine enzyme-cyclized streptavidin-binding motifs are advantageously used in combination with a (methyl)lanthionine-containing polypeptide of interest. For example, the polypeptide of interest is a member of a library of D,L-thioether-bridged peptides containing thioether bonds that bridge D- to L-amino acids or L- to D-amino acids, for example, a member of a hexa-, hepta- or octapeptide library.

In another embodiment, the proteinaceous substance comprises or consists of a non-naturally occurring (mutant) lantibiotic or a lantibiotic fragment comprising a ring structure, and wherein the ring structure comprises at least one cyclic streptavidin-binding motif containing a thioether bridge that bridges (in the orientation N- to C-) a D-amino acid to an L-amino acid, or an L-amino acid to a D-amino acid. More than 30 lantibiotics have been described in the art (Sahl et al., Annual Reviews in Microbiology 52, 41-79). They are grouped in two major categories based on their structural features and differences in their modes of action. Type A lantibiotics (e.g., nisin, epidermin and Pep5) are flexible, elongated, amphipathic molecules that mainly act by forming pores in the bacterial cytoplasmic membrane. Type B lantibiotics (e.g., mersacidin), in contrast, have a rigid globular-shaped end that inhibits particular enzymes. Lantibiotics are known in the art, and any of those known or yet to be discovered, or a fragment thereof, can be suitably modified, such that at least one of the rings contains an affinity tag. Examples include type A lantibiotics, such as nisin or subtilin, epidermin, gallidermin, mutacin 1140, mutacin I, mutacin B-Ny266, ericin A, ericin S, a fragment thereof, such as of nisin's rings ABC, and prenisin(1-45).

In one embodiment, the invention provides a mutant lantibiotic wherein ring A is mutated to comprise a cyclic affinity tag sequence, like an HPQ-containing streptavidin-binding sequence. It was surprisingly found that such alteration in the ring structure can be made without abolishing lantibiotic activity. In the nisin mutant G14H, A15P, L16Q, M17F, ΔG18 the five residues of ring C, GALMG (SEQ ID NO:40), have been replaced by four residues, HPQF (SEQ ID NO:7). This mutant, HPQF-nisin and also HPQF-nisin Δ(23-34) have growth-inhibiting effect of on the indicator strain MG1363. The specific activity is slightly lower than for nisin, but the production level is higher. Accordingly, provided is a mutant lantibiotic wherein at least ring A comprises an affinity tag, preferably wherein the amino acid sequence at positions 4-6 as in native nisin or subtilin is replaced by a streptavidin-binding sequence, like His-Pro-Gln (HPQ) (SEQ ID NO:5). Other potentially active HPQ-lanthionine-containing nisin variants have the HPQ sequence in ring A of nisin as in 14H, S5P, L6Q nisin, or having rings DE replaced by one ring that contains the sequence HPQF (SEQ ID NO:7) as in A24H, T25P, C26Q, H27F nisin, or lacticin A2 variants wherein residues 16-18 (TNT) (SEQ ID NO:41) in ring A are replaced by HPQ (SEQ ID NO:5). In a further embodiment, at least ring C comprises an affinity tag, preferably wherein the amino acid sequence at positions 14-17 as in native nisin or subtilin is replaced by the sequence His-Pro-Gln-Phe-Gly (HPQFG) (SEQ ID NO:42) or His-Pro-Gln-Phe (HPQF) (SEQ ID NO:7), preferably His-Pro-Gln-Phe (HPQF) (SEQ ID NO:7). In other exemplary mutant lantibiotics, at least rings D and E comprise an affinity tag sequence, preferably wherein the amino acid sequence at positions 24-27 as in native nisin or subtilin is replaced by the sequence His-Pro-Gln-Phe (SEQ ID NO:7) or His-Pro-Gln (SEQ ID NO:5).

The lantibiotic structure containing the cyclic affinity tag can be used advantageously as an external tag for any polypeptide of interest. However, it is particularly suitable to be used in combination with (e.g., fused to) a polypeptide, which in itself contains one or more thioether bridges, for example, a therapeutic protein genetically engineered to contain a (methyl)lanthionine to enhance its metabolic stability. By thioether ring stabilization, the peptides will require less frequent administration and lower doses, while the bioavailability and shelf life may be increased. The attachment of a thioether ring affinity tag facilitates the purification, detection and/or immobilization of such protein. For example, the cyclized proteinaceous substance is composed of a modified nisin sequence wherein ring C comprises a Streptag sequence, followed by a factor X cleavage site and a thioether-bridged Ang-(1-7) analog. Exemplary thioether-bridged Ang-(1-7) analogs include those disclosed in WO2008/018792. This molecule can be bound to a streptavidin column and released after washing as a purification step with higher yield and higher purity than by conventional hydrophobic interaction. Lantibiotics, or other peptides containing the engineered (methyl)lanthionine-streptavidin binding motif, can be efficiently purified using streptavidin columns, which are commercially available from several companies, for instance, GE Healthcare, following the protocol of the manufacturer keeping the pH not much higher than 7 (at alkaline pH, the dehydroresidues are unstable).

Also provided herein is a library comprising a multiplicity of proteinaceous substances comprising at least one cyclic tag sequence, e.g., a streptavidin-binding motif, the tag sequence being part of a motif containing a D,L- or an LD-thioether-linked ring structure that bridges (in the orientation N- to C-) a D-amino acid and an L-amino acid, or an L-amino acid to a D-amino acid. In addition to the tag sequence, the proteinaceous substance may contain one or more further thioether bridges. For instance, the library comprises a multiplicity of thioether-bridged peptides, each peptide furthermore comprising a lantibiotic enzyme-introduced thioether-cyclized Strep-tag. Each member of the library may be immobilized on a solid support, preferably in an array format. More preferably, each member is immobilized via the at least one cyclic tag sequence to a suitable (proteinaceous) binding partner spotted in an array format on a solid support. In a specific aspect, the invention provides a library of proteinaceous substances comprising a lantibiotic enzyme-cyclized Strep-tag sequence, each member being immobilized via the Strep-tag to streptavidin spotted in an array format on a solid support (strep-chip). Also within the scope of the invention is the use of such library for the identification of a peptide sequence of interest, for instance, a protein kinase substrate or a receptor ligand.

The skilled person will appreciate other useful applications hereof. For example, the thioether-bridged affinity tag may be used to purify, isolate and/or immobilize the polypeptide comprising the tag. Accordingly, also provided is a method for isolating, purifying and/or immobilizing a proteinaceous substance comprising a polypeptide of interest and a cyclic affinity tag, comprising the steps of:
a) providing at least one precursor proteinaceous substance, the precursor comprising the protein of interest and at least one motif of the general formula X1-Tag-X2 wherein
X1 and X2 represent amino acids whose side chains can be linked by a lantibiotic enzyme capable of forming a thioether bridge between residues X1 and X2;
Tag is an amino acid sequence serving as affinity tag when cyclized, the affinity tag allowing for capture of the proteinaceous substance to a specific binding partner. Typically, only the tag part of the proteinaceous substance mediates binding to the binding partner and other parts of the substance do not interact with the Tag-binding partner;
and wherein the motif is preceded N-terminally by a lantibiotic leader sequence;
b) contacting the precursor with at least one lantibiotic enzyme, allowing for the formation of a thioether bridge between X1 and X2, thereby introducing an intramolecular ring structure comprising the Tag sequence;
c) contacting the resulting cyclized proteinaceous substance with a specific capture reagent that binds the cyclized affinity tag with a dissociation constant less than about 10 μM, thereby isolating, purifying and/or immobilizing the substance. The capture reagent may be immobilized to a solid support, e.g., a column, an array surface, or a (magnetic) bead.

### LEGENDS TO THE FIGURES

FIG. 1: Enzymatic introduction of HPQF (SEQ ID NO:7) in ring C of nisin.

FIG. 1A: Mass spectrum of control peptide LMRTTSSLELSDYEQAC (SEQ ID NO:43) before (solid line) and after (dotted line) CDAP modification of a cysteine that yields 25 Da increase in mass.

FIG. 1B: Lactococcus lactis containing one plasmid encoding H14, P15, Q16, F17, ΔG18 prenisin and a second plasmid encoding NisB, NisT and NisC was induced and the supernatant was analyzed by mass spectrometry after addition of TCEP (solid line), which prevents disulfide formation, and CDAP (dotted line), which reacts with free cysteines. Absence of reactivity with CDAP proves lack of availability of the five cysteines and closure of all five thioether rings.

FIG. 2. Antimicrobial activity of a truncated nisin mutant containing HPQF (SEQ ID NO:7) in ring C. Lactococcus lactis containing a first plasmid encoding NisB, NisT and NisC and a second plasmid encoding MSTKDFNLDLVSVSKKDSGASPRITSISLCTPGCKTHPQFCNMKEQKLISEED (SEQ ID NO:44) was induced. The supernatant was treated with trypsin to cleave off the leader peptide. Series of two-fold dilutions were made in a microwell plate and the peptide's capacity to inhibit the growth of L. lactis MG1363 was tested by measuring OD 600 after six hours of incubation. ∘: control with PBS, ▪: truncated nisin mutant, ▾: supernatant of nisin A-producing L. lactis NZ9700.

### DETAILED DESCRIPTION OF THE INVENTION

### Experimental Section

### Example 1

### Replacement of the Ring C Sequence of Nisin by HPQF (SEQ ID NO:7)

### Materials and Methods

Control peptide was LMRTTSSLELSDYEQAC (SEQ ID NO:43). A nisin mutant was genetically made. In this H14, P15, Q16, F17, ΔG18 mutant nisin's ring C composed of GALMG (SEQ ID NO:40) was replaced by HPQF (SEQ ID NO:7). The encoding plasmid was co-expressed with pI13BTC (R. Rink et al., 2005, Biochemistry 44:8873-8882). Mass spec analyses was performed according to R. Rink et al., 2005, Biochemistry 44:8873-8882. Disulfide bridge formation was precluded by adding triscarboxyethyl phosphine (TCEP). Treatment with 1-cyano-4-dimethylaminopyridinium tetrafluoroborate (CDAP) was performed to measure the availability of cysteine. If a cysteine is not involved in thioether bridges, it is reactive with CDAP resulting in 25 Da upshift. Absence of 25 Da upshift of a TCEP- and CDAP-treated cysteine-containing peptide indicates the presence of a thioether bridge.

### Results

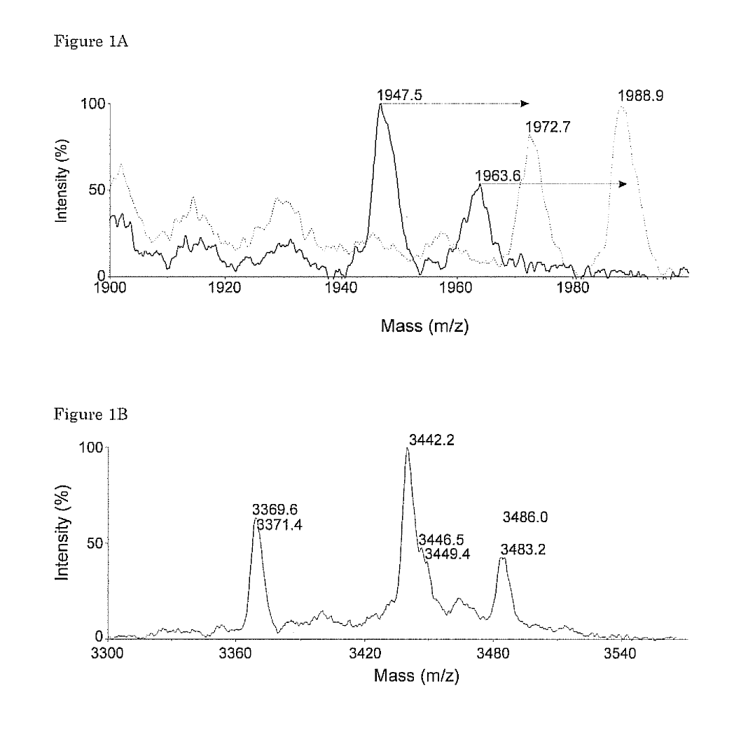

Average mass (m/z) of the unmodified control peptide was 1948.2 Da. Mass spectrometry revealed peaks of 1947.5 Da corresponding to the unmodified peptide and of 1963.6 Da, which might correspond to an oxidized form of this peptide; methionine2 might be candidate for the oxidation (FIG. 1A, solid line). In the control, peptide clearly peaks corresponding to the observed peaks with an increase of 25 Da corresponding to CDAP modification (FIG. 1A, dotted line). Clearly, the procedure proved that the cysteine in the control peptide was available for modification.

The expected mass of fully dehydrated prenisin mutant without methionine-1, containing HPQF (SEQ ID NO:7) was: 5911.8−144=5767.8 Da. Peptide with a mass of 5764.2 was observed in the supernatant (not shown). CDAP treatment did not lead to a 25 Da mass increase, as would have been the case for reaction of CDAP with free cysteines. Absence of reactivity indicated that all five cysteines were involved in thioether rings.

To increase the sensitivity of the mass spectrometry by removal of the leader peptide, the supernatant containing the fully dehydrated prenisin mutant comprising motif HPQF (SEQ ID NO:7) was treated with trypsin to cleave off the leader and was analyzed (FIG. 1B; solid line prior to CDAP addition, dotted line after CDAP treatment). The expected mass of this nisin mutant after full dehydration and removal of the leader is 3578.2−144=3434.2 Da. This value is close to the measured main peak in FIG. 1B of 3442 Da. The identity of the two other peaks, 3369 Da and 3486 Da, is not known. FIG. 1B demonstrates that addition of CDAP did not cause any shift in mass values. This again proves that all thioether rings were closed. Apparently, the shortened ring C containing HPQF (SEQ ID NO:7) instead of GALMG (SEQ ID NO:40) was very well formed.

### Conclusion

Example 1 demonstrates that HPQF (SEQ ID NO:7) can be introduced enzymatically by the cyclase NisC in ring C of nisin.

### Example 2

### Antimicrobial Activity of a Truncated Nisin Variant Containing an Affinity Tag in Ring C

### Materials and Methods

A truncated nisin mutant with internal strep-tag and external myc-tag was genetically constructed. The construct encoded the nisin leader peptide (MSTKDFNLDLVSVSKKDSGASPR (SEQ ID NO:45)) coupled to a H14, P15, Q16, F17, ΔG18 mutant of nisin (1-22), i.e., ITSISLCTPGCKTHPQFCNMK (SEQ ID NO:46), coupled to a myc-tag: EQKLISEED (SEQ ID NO:47). Hence the total presequence was MSTKDFNLDLVSVSKKDSGASPRITSISLCTPGCKTHPQFCNMKEQKLISEED (SEQ ID NO:44). The peptide was produced by L. lactis containing pI13BTC.

The leader was cleaved off with trypsin and the antimicrobial activity was measured as follows: In a microwell plate, 200 μl of supernatant of nisin fusion strain and in other wells, 200 μl of control (positive, e.g., filtered sup of NZ9700 and negative) solutions was added to the wells of the first column. 100 μl medium was added to the remaining empty wells. For each row, 100 μl from the first column was added to the second and, after mixing, from the second to the third, etc., leading to two-fold dilution steps. 100 μl of sensitive strain. MG1363 was added to all wells at low density, OD600: 0.05-0.1. After 6 hours, OD600 was measured.

### Results

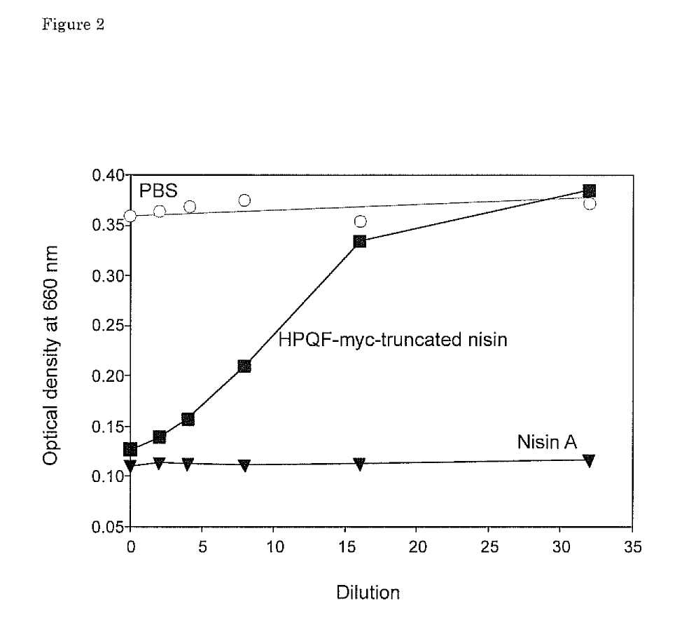

FIG. 2 shows that the truncated nisin's variant with HPQF-containing ring C clearly has antimicrobial activity.

### Conclusion

Example 2 demonstrates that the presence of HPQF (SEQ ID NO:7) in ring C does not abolish the antimicrobial activity of the truncated nisin mutant. This is surprising because four amino acids are simultaneously mutated, whereas one amino acid is deleted. Despite the truncation (which may already lead to a ten-fold reduction), despite the shortened ring C and despite the negatively charged tail (which reduces binding to the anionic membrane), the peptide still has activity.

1. An enzymatic method for providing a proteinaceous substance comprising a polypeptide of interest and a cyclic affinity tag, the method comprising the steps of:
a) providing at least one precursor proteinaceous substance, the precursor proteinaceous substance comprising said protein of interest and at least one motif of the general formula X1-Tag-X2 wherein
X1 and X2 represent amino acids whose side chains can be linked by a lantibiotic enzyme capable of forming a thioether bridge between residues X1 and X2;
Tag is an amino acid sequence serving as affinity tag when cyclised, said affinity tag allowing for capture of the proteinaceous substance to a specific binding partner of the tag; and
wherein said motif is preceded N-terminally by a lantibiotic leader sequence;
b) contacting said precursor proteinaceous substance with at least one lantibiotic enzyme, allowing for the formation of a thioether bridge between X1 and X2, thereby introducing an intramolecular ring structure comprising the Tag sequence; and
c) isolating the resulting cyclized proteinaceous substance.

2. The method according to claim 1, wherein said polypeptide of interest is fused N- or C-terminally to the at least one motif of the general formula X1-Tag-X2.

3. The method according to claim 2, wherein the proteinaceous substance comprises a cleavage site between said polypeptide of interest and the at least one motif.

4. The method according to claim 3, wherein step c) is followed by cleavage of the cyclized proteinaceous substance at the cleavage site to releasing the polypeptide of interest

5. The method according to claim 1, wherein said proteinaceous substance is a polypeptide of interest wherein a portion thereof is replaced by said at least one motif such that the motif is an integral part of said polypeptide of interest.

6. The method according to claim 1, wherein X1 is selected from the group consisting of Dhb, Dha, Thr, and Ser, and wherein X2 is Cys or Lys; or wherein X1 is Cys or Lys and X2 is selected from the group consisting of Dhb, Dha, Thr and Ser.

7. The method according to claim 1, wherein steps a) and b) are performed in a host cell comprising said at least one lantibiotic enzyme able to form a thioether-bridge bond between X1 and X2, said host cell being provided with a nucleic acid molecule encoding said precursor proteinaceous substance.

8. The method according to claim 1, wherein Tag comprises Arg-Gly-Asp.

9. The method according to claim 1, wherein Tag comprises a streptavidin binding molecule able to bind streptavidin with a dissociation constant less than 10 μM.

10. The method according to claim 9, wherein the motif X1-Tag-X2 consists of an amino acid molecule selected from the group consisting of Dha-His-Pro-Gln-Phe-Cys (Dha-SEQ ID NO:48; Dhb-His-Pro-Gln-Phe-Cys (Dhb-SEQ ID NO:48); Ser-His-Pro-Gln-Phe-Cys (SEQ ID NO:24; Thr-His-Pro-Gln-Phe-Cys (SEQ ID NO:25; Cys-His-Pro-Gln-Phe-Dha (SEQ ID NO:49-Dha); Cys-His-Pro-Gln-Phe-Dhb (SEQ ID NO:49-Dhb); Cys-His-Pro-Gln-Phe-Ser (SEQ ID NO:26); Cys-His-Pro-Gln-Phe-Thr (SEQ ID NO:27); Dha-His-Pro-Gln-Cys (Dha-SEQ ID NO:50); Dhb-His-Pro-Gln-Cys (Dhb-SEQ ID NO:50); Ser-His-Pro-Gln-Cys (SEQ ID NO:28); Thr-His-Pro-Gln-Cys (SEQ ID NO:29); Cys-His-Pro-Gln-Dha (SEQ ID NO:51-Dha); Cys-His-Pro-Gln-Dhb (SEQ ID NO:51-Dhb); Cys-His-Pro-Gln-Ser (SEQ ID NO:30); Cys-His-Pro-Gln-Thr (SEQ ID NO:31); Ser-His-Pro-Gln-Phe-Lys (SEQ ID NO:32);
Thr-His-Pro-Gln-Phe-Lys (SEQ ID NO:33); Lys-His-Pro-Gln-Phe-Ser (SEQ ID NO:34); Lys-His-Pro-Gln-Phe-Thr (SEQ ID NO:35); Dha-His-Pro-Gln-Phe-Lys (Dha-SEQ ID NO:52); Dhb-His-Pro-Gln-Phe-Lys (Dhb-SEQ ID NO:52); Lys-His-Pro-Gln-Dha (SEQ ID NO:53-Dha); Lys-His-Pro-Gln-Dhb (SEQ ID NO:53-Dhb); Ser-His-Pro-Gln-Lys (SEQ ID NO:36); Thr-His-Pro-Gln-Lys (SEQ ID NO:37); Lys-His-Pro-Gln-Ser (SEQ ID NO:38); Lys-His-Pro-Gln-Thr (SEQ ID NO:39); Dha-His-Pro-Gln-Lys (Dha-SEQ ID NO:54); and Dhb-His-Pro-Gln-Lys (Dhb-SEQ ID NO:54); Lys His Pro Gln Dha; and Lys His Pro Gln Dhb.

11. The method according to claim 1, wherein step b) comprising contacting the precursor with lantibiotic enzyme LanM, cyclase LanC (in the case of a combination of a dehydroresidue and a cysteine) or a combination of a lantibiotic dehydratase LanB and cyclase LanC.

12. The method according to claim 1, wherein the polypeptide of interest naturally comprises at least one thioether-containing intramolecular ring structure.

13. Proteinaceous substance comprising a cyclic tag sequence, produced by the method according to claim 1.

14. A proteinaceous substance comprising at least one cyclic tag sequence, the tag sequence being part of a thioether-linked ring structure bridging (in the orientation N- to C-) a D-amino acid and an L-amino acid, or an L-amino acid to a D-amino acid.

15. Proteinaceous substance according to claim 14, wherein said cyclic tag sequence is cyclized streptavidin binding sequence.

16. Proteinaceous substance according to claim 14, comprising a mutant lantibiotic or a lantibiotic fragment comprising a ring structure, and wherein said ring structure comprises at least one cyclic streptavidin binding motif containing a thioether bridge, that bridges (in the orientation N- to C-) a D-amino acid to an L-amino acid, or an L-amino acid to a D-amino acid.

17. A peptide library comprising a multiplicity of proteinaceous substances according to claim 14.

18. The peptide library of claim 17, wherein each member is immobilized on a solid support.

19. The peptide library of claim 18, comprising a multiplicity of proteinaceous substances comprising at least one cyclic streptavidin binding motif, wherein each member is immobilized via the at least one cyclic streptavidin binding motif to streptavidin spotted in an array format on a solid support.

20. The method according to claim 3, wherein the cleavage site is a Factor X or a Glu-C-cleavage site.

21. The method according to claim 9, wherein the streptavidin binding molecule is selected from the group consisting of His-Pro-Gly (HPG), His-Pro-Lys (HPK), His-Pro-Met (HPM), His-Pro-Gln (HPQ), and His-Pro-Gln-Phe (HPQF) (SEQ ID NO:7).

22. The method according to claim 9, wherein the streptavidin binding molecule is selected from the group consisting of His-Pro-Gln (HPQ) or His-Pro-Gln-Phe (HPQF).

23. The method according to claim 11, wherein steps a) and b) are performed in a host cell comprising lanthionine proteins
LanB;
LanC and LanT;
LanM and LanT;
LanB and LanC; or
LanM.

24. The proteinaceous substance of claim 15, wherein the cyclized streptavidin binding molecule comprises
His-Pro-Gly,
His-Pro-Lys,
His-Pro-Met,
His-Pro-Gln, and
His-Pro-Gln-Phe (SEQ ID NO:7).

25. The proteinaceous substance of claim 15, wherein the cyclized streptavidin binding molecule consists of
His-Pro-Gly,
His-Pro-Lys,
His-Pro-Met,
His-Pro-Gln, and
His-Pro-Gln-Phe (SEQ ID NO:7).

26. The peptide library of claim 18, wherein each member is immobilized on a solid support in an array format.


SEQUENCE LISTING (55 sequences)

SEQ ID NO: 1 - protein, 3 aa, Artificial
  Tag sequence
RGD

SEQ ID NO: 2 - protein, 3 aa, Artificial
  Tag sequence
HPG

SEQ ID NO: 3 - protein, 3 aa, Artificial
  Tag sequence
HPK

SEQ ID NO: 4 - protein, 3 aa, Artificial
  Tag sequence
HPM

SEQ ID NO: 5 - protein, 3 aa, Artificial
  Tag sequence
HPQ

SEQ ID NO: 6 - protein, 3 aa, Artificial
  Tag sequence
HPN

SEQ ID NO: 7 - protein, 4 aa, Artificial
  Tag sequence
HPQF

SEQ ID NO: 8 - protein, 5 aa, Artificial
  Tag sequence
DVEAW

SEQ ID NO: 9 - protein, 6 aa, Artificial
  Tag sequence
DVEAWX

SEQ ID NO: 10 - protein, 4 aa, Artificial
  Tag sequence
DVEA

SEQ ID NO: 11 - protein, 4 aa, Artificial
  Tag sequence
VEAW

SEQ ID NO: 12 - protein, 3 aa, Artificial
  Tag sequence
DVE

SEQ ID NO: 13 - protein, 3 aa, Artificial
  Tag sequence
VEA

SEQ ID NO: 14 - protein, 3 aa, Artificial
  Tag sequence
EAW

SEQ ID NO: 15 - protein, 6 aa, Artificial
  Tag sequence
VPLVET

SEQ ID NO: 16 - protein, 5 aa, Artificial
  Tag sequence
DVXAW

SEQ ID NO: 17 - protein, 5 aa, Artificial
  Tag sequence
EPDWX

SEQ ID NO: 18 - protein, 5 aa, Artificial
  Tag sequence
GDXXF

SEQ ID NO: 19 - protein, 5 aa, Artificial
  Tag sequence
PWXWL

SEQ ID NO: 20 - protein, 4 aa, Artificial
  Tag sequence
VPEY

SEQ ID NO: 21 - protein, 4 aa, Artificial
  conserved leader sequence box
FNLD

SEQ ID NO: 22 - protein, 3 aa, Artificial
  conserved leader sequence box
ELD

SEQ ID NO: 23 - protein, 3 aa, Artificial
  conserved leader sequence box
EEV

SEQ ID NO: 24 - protein, 6 aa, Artificial
  Tag motif
SHPQFC

SEQ ID NO: 25 - protein, 6 aa, Artificial
  Tag motif
THPQFC

SEQ ID NO: 26 - protein, 6 aa, Artificial
  tag motif
CHPQFS

SEQ ID NO: 27 - protein, 6 aa, Artificial
  Tag motif
CHPQFT

SEQ ID NO: 28 - protein, 5 aa, Artificial
  Tag motif
SHPQC

SEQ ID NO: 29 - protein, 5 aa, Artificial
  Tag motif
THPQC

SEQ ID NO: 30 - protein, 5 aa, Artificial
  tag motif
CHPQS

SEQ ID NO: 31 - protein, 5 aa, Artificial
  tag motif
CHPQT

SEQ ID NO: 32 - protein, 6 aa, Artificial
  Tag motif
SHPQFK

SEQ ID NO: 33 - protein, 6 aa, Artificial
  Tag motif
THPQFK

SEQ ID NO: 34 - protein, 6 aa, Artificial
  Tag motif
KHPQFS

SEQ ID NO: 35 - protein, 6 aa, Artificial
  Tag motif
KHPQFT

SEQ ID NO: 36 - protein, 5 aa, Artificial
  tag motif
SHPQK

SEQ ID NO: 37 - protein, 5 aa, Artificial
  Tag motif
THPQK

SEQ ID NO: 38 - protein, 5 aa, Artificial
  tag motif
KHPQS

SEQ ID NO: 39 - protein, 5 aa, Artificial
  tag motif
KHPQT

SEQ ID NO: 40 - protein, 5 aa, Artificial
  ring C residues of nisin
GALMG

SEQ ID NO: 41 - protein, 3 aa, Artificial
  ring A residues of lacticin
TNT

SEQ ID NO: 42 - protein, 5 aa, Artificial
  tag
HPQFG

SEQ ID NO: 43 - protein, 17 aa, Artificial
  control peptide
LMRTTSSLELSDYEQAC

SEQ ID NO: 44 - protein, 53 aa, Artificial
  nisin mutant peptide
MSTKDFNLDLVSVSKKDSGASPRITSISLCTPGCKTHPQFCNMKEQKLISEED

SEQ ID NO: 45 - protein, 23 aa, Artificial
  truncated nisin
MSTKDFNLDLVSVSKKDSGASPR

SEQ ID NO: 46 - protein, 21 aa, Artificial
  nisin mutant
ITSISLCTPGCKTHPQFCNMK

SEQ ID NO: 47 - protein, 9 aa, Artificial
  myc tag
EQKLISEED

SEQ ID NO: 48 - protein, 5 aa, Artificial
  tag motif
HPQFC

SEQ ID NO: 49 - protein, 5 aa, Artificial
  tag motif
CHPQF

SEQ ID NO: 50 - protein, 4 aa, Artificial
  tag motif
HPQC

SEQ ID NO: 51 - protein, 4 aa, Artificial
  tag motif
CHPQ

SEQ ID NO: 52 - protein, 5 aa, Artificial
  tag motif
HPQFK

SEQ ID NO: 53 - protein, 4 aa, Artificial
  tag motif
KHPQ

SEQ ID NO: 54 - protein, 4 aa, Artificial
  tag motif
HPQK

SEQ ID NO: 55 - protein, 6 aa, Artificial
  tag motif
HPQFGC